(19) United States
(12) Patent Application Publication
(10) Pub. No.: US 2025/0121020 A1
(43) Pub. Date: Apr. 17, 2025

(54) COMPOSITION FOR IMPROVING MEMORY AND PREVENTING, ALLEVIATING, OR TREATING COGNITIVE DISORDER COMPRISING YUKGUNJATANG AS EFFECTIVE COMPONENT

(71) Applicant: KOREA INSTITUTE OF ORIENTAL MEDICINE, Daejeon (KR)

(72) Inventors: MALK EUN PAK, Daegu (KR); YOUNGHOON GO, Gyeongsangbuk-do (KR); WEI LI, Seoul (KR); HYE JIN YANG, Daejeon (KR)

(21) Appl. No.: 18/989,473

(22) Filed: Dec. 20, 2024

Related U.S. Application Data
(63) Continuation-in-part of application No. PCT/KR2023/007407, filed on May 31, 2023.

(30) Foreign Application Priority Data
Jun. 21, 2022 (KR) 10-2022-0075397

(51) Int. Cl. A61K 36/752; A23K 20/10; A61K 36/076; A61K 36/258; A61K 36/284; A61K 36/484; A61K 36/725; A61K 36/8888; A61K 36/9068; A61P 25/28

(52) U.S. Cl. CPC A61K 36/752; A23K 20/10; A61K 36/076; A61K 36/258; A61K 36/284; A61K 36/484; A61K 36/725; A61K 36/8888; A61K 36/9068; A61P 25/28

(57) ABSTRACT

A composition including Yukgunjatang as effective component is effective for improving memory and preventing, alleviating, or treating cognitive disorder. Yukgunjatang, which is prepared by boiling a mixture of Gingseng Radix, Atractylodes rhizoma alba, Hoelen, Glycyrrhizae Radix, Aurantii Nobilis Pericarpium, Pinelliae Rhizoma, Zingiberis Rhizoma, and Zizyphi Fructus in water, exhibits superior neuroprotective effect compared to individual extracts of Gingseng Radix, Atractylodes rhizoma alba, Hoelen, Glycyrrhizae Radix, Aurantii Nobilis Pericarpium, Pinelliae Rhizoma, Zingiberis Rhizoma, and Zizyphi Fructus, and, in an animal model of cognitive decline induced by scopolamine, administration of Yukgunjatang shows the effect of improving memory and cognitive function. Thus, the composition can be advantageously used as a food product, a medicinal product, or the like for preventing or treating brain diseases including Alzheimer's disease, Parkinson's disease, and mild cognitive impairment.

### CROSS-REFERENCE TO RELATED APPLICATION AND CLAIM OF PRIORITY

The present application is a continuation in part of application to International Application No. PCT/KR2023/007407 with an International Filing Date of May 31, 2023, which claims the benefit of Korean Patent Application No. 10-2022-0075397 filed on Jun. 21, 2022 at the Korean Intellectual Property Office, the disclosures of which are incorporated by reference herein in their entirety.

### BACKGROUND

### 1. Technical Field

The present invention relates to a composition for improving memory and preventing, alleviating, or treating cognitive disorder comprising Yukgunjatang as effective component.

This work is supported by the major project fund of the Ministry of Science and ICT, South Korea (Project Numbers: 1711150203 and 1711195903).

### 2. Background Art

As the social environment rapidly changes and diversifies, modern people must engage in excessive brain activity to absorb a lot of information and adapt quickly. In particular, students preparing for competitive entrance exams have to engage in prolonged learning and memory activities due to fierce competition. After middle age, cognitive decline in the central nervous system can lead to memory loss, forgetfulness, and increased anxiety. In addition, in the elderly, degenerative diseases such as dementia deteriorate memory, and as the condition worsens, not only does it make social life impossible, but also does it become a disease that requires enormous social costs considering the impact it has on family members and people around them.

Among methods for improving memory or preventing memory decline related to brain tissue, surgical treatment, medication, or the intake of functional substances are commonly mentioned. Since surgical treatments are not easily accessible, diseases like dementia that involve memory decline are better managed through prevention than treatment after the onset of a disease. This is achieved by consistently consuming medications or functional substances that are effective in improving memory or preventing its decline. Therefore, there is a growing need to develop methods that utilize natural products, which are safe, free from side effects, and suitable for continuous consumption, to effectively enhance memory.

Meanwhile, Yukgunjatang (i.e., Rikkunshito) is a traditional herbal prescription formula comprised of Atractylodes rhizoma alba, Gingseng Radix, Hoelen, Glycyrrhizae Radix, Aurantii Nobilis Pericarpium (Fruit peel), Pinelliae Rhizoma, Zingiberis Rhizoma, and Zizyphi Fructus. This prescription formula was first documented in the Gyoyubuinbang (Treatise on Obstetrics and Gynecology) by Jin Ziming during the Song Dynasty. In modern days, it is known to be effective for treating digestive disorders such as chronic gastritis, gastroptosis, and gastric atony.

Prior art related to the enhancement of memory or the improvement of cognitive impairment includes Korean Patent Registration No. 2065503, which discloses “Composition for prevention or treatment of learning disabilities, cognitive impairments, or memory loss containing a fraction of ginseng extract,” and Korean Patent Publication No. 2005-0092292, which discloses “Herbal composition with memory-enhancing effects.” However, there has been no disclosure to date regarding the composition of the present invention, specifically “Composition for improving memory and preventing, alleviating, or treating cognitive disorder comprising Yukgunjatang as effective component.”

### SUMMARY

The present invention is devised in view of the need described above, and provided by the present invention is a composition for improving memory and preventing, alleviating, or treating cognitive disorder comprising Yukgunjatang as effective component. Specifically, it is found that Yukgunjatang, which is prepared by boiling a mixture of Gingseng Radix, Atractylodes rhizoma alba, Hoelen, Glycyrrhizae Radix, Aurantii Nobilis Pericarpium, Pinelliae Rhizoma, Zingiberis Rhizoma, and Zizyphi Fructus in water, exhibits superior neuroprotective effect compared to individual extracts of Gingseng Radix, Atractylodes rhizoma alba, Hoelen, Glycyrrhizae Radix, Aurantii Nobilis Pericarpium, Pinelliae Rhizoma, Zingiberis Rhizoma, and Zizyphi Fructus, and, in an animal model of cognitive decline induced by scopolamine, administration of Yukgunjatang leads to the improvement of memory and cognitive function. The present invention is completed accordingly.

To solve the problems that are described in the above, the present invention provides a functional health food composition for improving cognitive function or memory comprising Yukgunjatang as effective component.

The present invention further provides a pharmaceutical composition for preventing or treating degenerative brain disease comprising Yukgunjatang as effective component.

The present invention still further provides an animal feed additive composition for preventing or ameliorating degenerative brain disease comprising Yukgunjatang as effective component.

The present invention relates to a composition for improving memory and preventing, alleviating, or treating cognitive disorder comprising Yukgunjatang as effective component. Specifically, it is found that Yukgunjatang, which is prepared by boiling a mixture of Gingseng Radix, Atractylodes rhizoma alba, Hoelen, Glycyrrhizae Radix, Aurantii Nobilis Pericarpium, Pinelliae Rhizoma, Zingiberis Rhizoma, and Zizyphi Fructus in water, exhibits superior neuroprotective effect compared to individual extracts of Gingseng Radix, Atractylodes rhizoma alba, Hoelen, Glycyrrhizae Radix, Aurantii Nobilis Pericarpium, Pinelliae Rhizoma, Zingiberis Rhizoma, and Zizyphi Fructus, and, in an animal model of cognitive decline induced by scopolamine, administration of Yukgunjatang shows the effect of improving memory and cognitive function.

### BRIEF DESCRIPTION OF THE DRAWINGS

FIG. 1 illustrates the result of determining cell viability after treating mouse-derived hippocampus neuronal cells with Yukgunjatang or individual extracts of Gingseng Radix, Pinelliae Rhizoma, Atractylodes rhizoma alba, Hoelen, Aurantii Nobilis Pericarpium, Glycyrrhizae Radix, Zingiberis Rhizoma, and Zizyphi Fructus, which constitute Yukgunjatang. ## indicates that, compared to the control group (Con), the cell viability significantly increased in the groups treated with the individual extracts of Gingseng Radix, Pinelliae Rhizoma, Atractylodes rhizoma alba, Hoelen, Aurantii Nobilis Pericarpium, Glycyrrhizae Radix, Zingiberis Rhizoma, and Zizyphi Fructus, as well as in the group treated with Yukgunjatang. The increase was statistically significant, with p<0.01.

FIG. 2 illustrates the result of determining neuroprotective effect after treating mouse-derived hippocampus neuronal cells with Yukgunjatang or individual extracts of Gingseng Radix, Pinelliae Rhizoma, Atractylodes rhizoma alba, Hoelen, Aurantii Nobilis Pericarpium, Glycyrrhizae Radix, Zingiberis Rhizoma, and Zizyphi Fructus, which constitute Yukgunjatang. $$ indicates that, compared to the glutamate treatment group (−), the cell viability significantly increased in the group treated with both glutamate and Yukgunjatang (100 μg/mL), in which the increase was statistically significant, with p<0.01. * and ** indicate that the cell viability increased significantly in the group treated with glutamate and Yukgunjatang, comparted to the groups treated with glutamate and the individual extracts of Gingseng Radix, Pinelliae Rhizoma, Atractylodes rhizoma alba, Hoelen, Aurantii Nobilis Pericarpium, Glycyrrhizae Radix, Zingiberis Rhizoma, and Zizyphi Fructus (each at 100 μg/mL), which constitute Yukgunjatang, in which the increase was statistically significant, with p<0.05 for * and p<0.01 for **.

FIG. 3 illustrates the result of evaluating the degree of cell death after co-treatment with glutamate (GLU) and Yukgunjatang (YGT). ### indicates that the degree of cell death in the glutamate treatment group (−) significantly increased compared to the normal group (Con), in which the increase was statistically significant, with p<0.001. Meanwhile, * and *** indicate that the degree of cell death significantly decreased in the group treated with glutamate and Yukgunjatang at 50 μg/mL or 100 μg/mL, compared to the glutamate treatment group (−), in which the decrease was statistically significant, with p<0.05 for * and p<0.001 for ***.

FIG. 4 is a schematic diagram of an animal test that shows the cognitive function improvement effect of the administration of Yukgunjatang of the present invention in an animal model with scopolamine-induced cognitive decline. NORT refers to the Novel Object Recognition Test, and PAT refers to the Passive Avoidance Test.

FIG. 5 illustrates the result of measuring the memory index following the administration of Yukgunjatang (YGT) in a scopolamine-induced cognitive decline animal model. Con represents the normal group, SCO is the control group treated with scopolamine, and SCO+YGT is the group treated with both scopolamine and Yukgunjatang. ## indicates that the memory index of the scopolamine-treated control group (SCO) was significantly decreased compared to the normal group (Con), in which the decrease was statistically significant, with p<0.01. ** indicates that the memory index of the SCO+YGT group (treated with both scopolamine and Yukgunjatang) was statistically significantly increased compared to the control group (SCO) which has been treated only with scopolamine, with p<0.01.

FIG. 6 illustrates the result of measuring the latency (i.e. stay time) following the administration of Yukgunjatang (YGT) in a scopolamine-induced cognitive decline animal model. Con represents the normal group, SCO is the control group treated with scopolamine, and SCO+YGT is the group treated with both scopolamine and Yukgunjatang. ### indicates that the latency of the scopolamine-treated control group (SCO) was significantly decreased compared to the normal group (Con), in which the decrease was statistically significant, with p<0.001. * indicates that the latency of the SCO+YGT group (treated with both scopolamine and Yukgunjatang) was statistically significantly increased compared to the control group (SCO) which has been treated only with scopolamine, with p<0.05.

### DETAILED DESCRIPTION

To achieve the object that is described in the above, the present invention provides a functional health food composition for improving cognitive function or memory comprising Yukgunjatang as effective component.

The Yukgunjatang is preferably prepared by boiling a mixture of Gingseng Radix, Atractylodes rhizoma alba, Hoelen, Glycyrrhizae Radix, Aurantii Nobilis Pericarpium, Pinelliae Rhizoma, Zingiberis Rhizoma, and Zizyphi Fructus in water, but it is not limited to those prepared by this method.

According to one embodiment of the present invention, Yukgunjatang may be prepared by boiling a mixture of 0.6 to 2.2 parts by weight of Gingseng Radix, 0.6 to 2.2 parts by weight of Atractylodes rhizoma alba, 0.6 to 2.2 parts by weight of Hoelen, 0.6 to 2.2 parts by weight of Pinelliae Rhizoma, 0.6 to 2.2 parts by weight of Aurantii Nobilis Pericarpium, 0.2 to 0.8 part by weight of Glycyrrhizae Radix, 0.3 to 1.0 part by weight of Zingiberis Rhizoma, and 0.3 to 1.0 part by weight of Zizyphi Fructus in water. It may be preferably prepared by boiling a mixture of 0.8 to 1.6 parts by weight of Gingseng Radix, 0.8 to 1.6 parts by weight of Atractylodes rhizoma alba, 0.8 to 1.6 parts by weight of Hoelen, 0.8 to 1.6 parts by weight of Pinelliae Rhizoma, 0.8 to 1.6 parts by weight of Aurantii Nobilis Pericarpium, 0.3 to 0.7 part by weight of Glycyrrhizae Radix, 0.5 to 0.8 part by weight of Zingiberis Rhizoma, and 0.5 to 0.8 part by weight of Zizyphi Fructus in water, or most preferably prepared by boiling a mixture of 1.33 parts by weight of Gingseng Radix, 1.33 parts by weight of Atractylodes rhizoma alba, 1.33 parts by weight of Hoelen, 1.33 parts by weight of Pinelliae Rhizoma, 1.33 parts by weight of Aurantii Nobilis Pericarpium, 0.5 part by weight of Glycyrrhizae Radix, 0.67 part by weight of Zingiberis Rhizoma, and 0.67 part by weight of Zizyphi Fructus in water, but it is not limited thereto.

The composition may be prepared in any one of formulations selected from powder, granule, pill, tablet, capsule, candy, syrup, and beverage, but it is not limited to these formulations.

When the functional health food composition of the present invention is used as a food additive, it may be added as is or used together with other foods or food ingredients, and it can be used appropriately according to conventional methods. The amount of effective component can be adjusted depending on its intended purpose (prevention or improvement). Generally, when manufacturing food or beverages, the functional health food composition of the present invention is added in an amount not exceeding 15 parts by weight of the total ingredients, and preferably not exceeding 10 parts by weight. However, for long-term consumption for health purposes, the amount may be below the specified range, and since there are no safety concerns, the effective components can be used in amounts exceeding the specified range.

There are no specific limitations on the types of functional health foods. Examples of foods to which the functional health food composition can be added include meat, sausages, bread, chocolates, candies, snacks, confectioneries, pizza, ramen, noodles, gum, dairy products such as ice cream, various soups, beverages, tea drinks, alcoholic beverages, and vitamin supplements, as well as any health foods in the general sense.

Additionally, the functional health food composition of the present invention can be manufactured as a food, particularly as a functional food. The functional food of the present invention includes ingredients commonly added during food manufacturing, such as proteins, carbohydrates, fats, nutrients, and seasonings. For example, when manufactured as a drink, natural carbohydrates or flavoring agents may be included as additional ingredients, in addition to the effective components. The natural carbohydrates are preferably monosaccharides (e.g., glucose, fructose), disaccharides (e.g., maltose, sucrose), oligosaccharides, polysaccharides (e.g., dextrin, cyclodextrin), or sugar alcohols (e.g., xylitol, sorbitol, erythritol). The flavoring agents can include natural flavoring agents (e.g., thaumatin, stevia extract) and synthetic flavoring agents (e.g., saccharin, aspartame).

In addition to the functional health food composition, various supplements, vitamins, electrolytes, flavoring agents, colorants, pectinic acid and its salts, alginic acid and its salts, organic acids, protective colloid thickeners, pH adjusters, stabilizers, preservatives, glycerin, alcohol, carbonation agents used in carbonated beverages, and the like may also be included. The ratio of these additional ingredients is not critically important, but it is generally selected within the range of 0.01 to 0.1 part by weight with respect to 100 parts by weight of the functional health food composition of the present invention.

The present invention further provides a pharmaceutical composition for preventing or treating degenerative brain disease comprising Yukgunjatang as effective component.

The degenerative brain disease referred to in the present invention is preferably Alzheimer's disease, mild cognitive impairment, or dementia, but is not limited to these conditions.

The pharmaceutical composition according to the present invention can be formulated into oral forms such as capsules, powders, granules, tablets, suspensions, emulsions, syrups, and aerosols, as well as external preparations, suppositories, and sterile injectable solutions using conventional methods.

The pharmaceutical composition according to the present invention may further include pharmaceutically acceptable carriers, excipients, or diluents.

The carriers, excipients, and diluents that may be included in the pharmaceutical composition of the present invention include various compounds or mixtures such as lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil.

When formulating, the pharmaceutical composition is prepared using common diluents or excipients such as fillers, bulking agents, binders, wetting agents, disintegrants, and surfactants. Solid dosage forms for oral administration include tablets, pills, powders, granules, and capsules. These solid dosage forms are typically prepared by mixing at least one excipient, such as starch, calcium carbonate, sucrose, lactose, or gelatin with the pharmaceutical composition of the present invention. Additionally, lubricants like magnesium stearate and talc may also be used alongside simple excipients. For liquid dosage forms for oral administration, suspensions, solutions, emulsions, and syrups are usable. Commonly used diluents such as water and liquid paraffin, as well as various excipients like wetting agents, sweeteners, flavoring agents, and preservatives, may be included. For parenteral dosage forms, preparations include sterile aqueous solutions, non-aqueous solutions, suspensions, emulsions, freeze-dried formulations, and suppositories. Non-aqueous solutions and suspensions can use propylene glycol, polyethylene glycol, plant oils like olive oil, or injectable esters like ethyl oleate. As suppository bases, materials such as Witepsol, Macrogol, Tween 61, cocoa butter, laurin fat, and glycerogelatin can be used.

The appropriate dosage of the pharmaceutical composition of the present invention may vary depending on various factors such as the formulation method, administration route, patient's age, weight, gender, pathological condition, diet, administration time, administration route, excretion rate, and responsiveness.

The pharmaceutical composition of the present invention can be administered either orally or parenterally. In the case of parenteral administration, it can be administered topically on the skin, by intravenous injection, subcutaneous injection, intramuscular injection, intraperitoneal injection, or transdermal delivery.

The present invention still further provides an animal feed additive composition for preventing or ameliorating degenerative brain disease comprising Yukgunjatang as effective component.

The feed additive of the present invention corresponds to an auxiliary feed as defined in the Feed Management Act. In the present invention, the term ‘feed’ refers to any natural or artificial food, such as a formulated diet, a single meal, or the components of such meals, that animals eat, ingest, and digest. The types of feed are not particularly limited, and any feed commonly used in the relevant technical field can be used. Non-limiting examples of the feed include plant-based feeds such as grains, tubers, food processing byproducts, seaweeds, fiber sources, pharmaceutical byproducts, oils and fats, starches, or grain byproducts; and proteins, inorganic materials, oils and fats, minerals, single-cell proteins, and animal-based feeds such as animal plankton or food. These can be used either alone or in combination of two or more of them.

Hereinbelow, the present invention is explained in greater detail in view of Examples. However, the following Examples are given only for exemplification of the present invention and it is evident that the scope of the present invention is not limited by them.

### EXAMPLES

### [Preparation of Yukgunjatang]

Yukgunjatang used in the present invention is an extract of composite oriental medicine which consists of 1.33 g of Gingseng Radix, 1.33 g of Atractylodes rhizoma alba, 1.33 g of Hoelen, 1.33 g of Pinelliae Rhizoma, 1.33 g of Aurantii Nobilis Pericarpium, 0.5 g of Glycyrrhizae Radix, 0.67 g of Zingiberis Rhizoma, and 0.67 g of Zizyphi Fructus, and it was purchased from Hankookshinyak Pharmaceutical Co., Ltd.

### [Preparation of Individual Extracts]

To prepare each of the individual extracts of the constituents of Yukgunjatang, Gingseng Radix, Atractylodes rhizoma alba, Hoelen, Pinelliae Rhizoma, Aurantii Nobilis Pericarpium, Glycyrrhizae Radix, Zingiberis Rhizoma, and Zizyphi Fructus were purchased from Omniherb (Dongwoodang). For each of the individual extracts, 100 g of the sample were subjected to hot water extraction after adding them to 1 liter of hot water. The individual extracts were then freeze-dried until use.

### Example 1. Neuroprotective Effect

The test was conducted using the HT22 cell line, a mouse hippocampus neuronal cell line separated from hippocampus, which is responsible for cognitive function. After the application on a plate, the HT22 neuronal cells were stabilized for 24 hours. Subsequently, they were treated with samples (i.e., individual extracts of Gingseng Radix, Atractylodes rhizoma alba, Hoelen, Pinelliae Rhizoma, Aurantii Nobilis Pericarpium, Glycyrrhizae Radix, Zingiberis Rhizoma, and Zizyphi Fructus, as well as Yukgunjatang) at a concentration of 100 μg/mL and cultured for 24 hours. Subsequently, the cells were co-treated with the sample and glutamate, which induces neuronal cell damage, and cultured for 24 hours. After that, cell viability was assessed using a CCK-8 assay, while cell death was evaluated by flow cytometry analysis following Annexin V/PI staining.

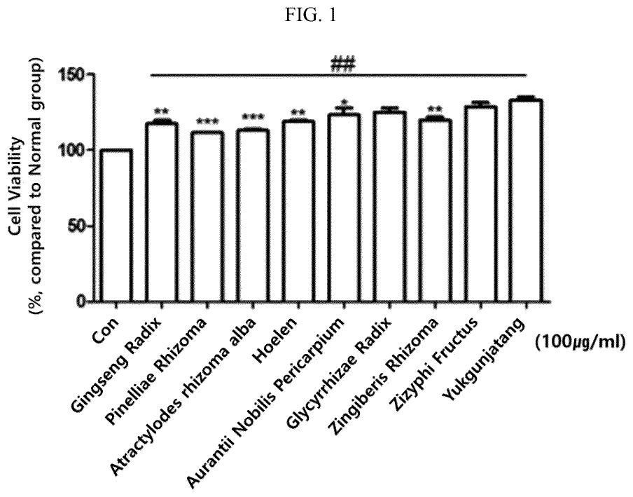

As shown in FIG. 1, the results indicated that neither Yukgunjatang nor the individual extracts of its constituents, i.e., Gingseng Radix, Atractylodes rhizoma alba, Hoelen, Pinelliae Rhizoma, Aurantii Nobilis Pericarpium, Glycyrrhizae Radix, Zingiberis Rhizoma, and Zizyphi Fructus, exhibited any cytotoxicity.

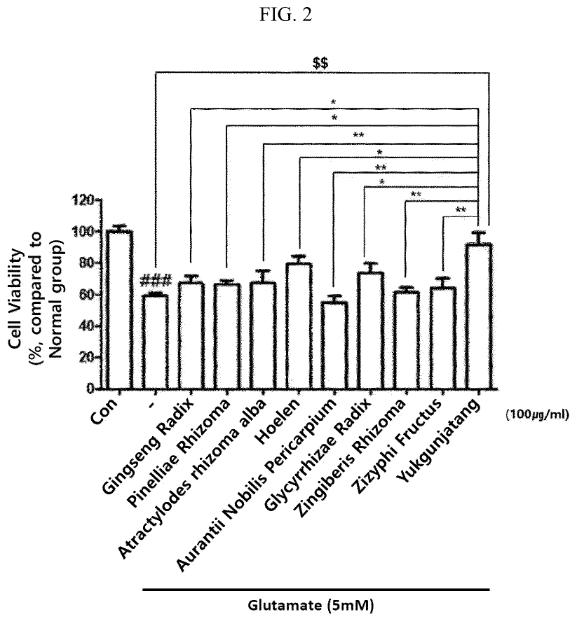

Furthermore, with regard to the glutamate-induced cytotoxicity, the cell viability increased when Yukgunjatang was co-administered. Notably, Yukgunjatang showed a statistically significant greater neuroprotective effect compared to the individual extracts of its components, i.e., Gingseng Radix, Atractylodes rhizoma alba, Hoelen, Pinelliae Rhizoma, Aurantii Nobilis Pericarpium, Glycyrrhizae Radix, Zingiberis Rhizoma, and Zizyphi Fructus (FIG. 2).

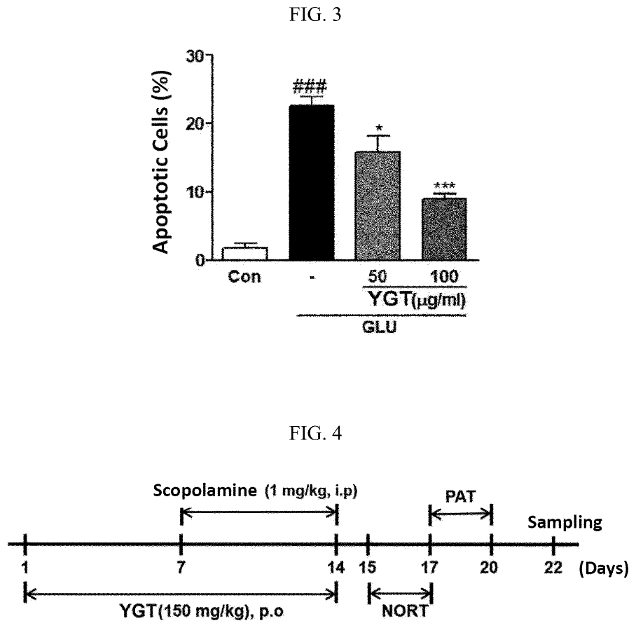

Furthermore, the increased neuronal cell death induced by glutamate treatment showed a statistically significant reduction when Yukgunjatang was co-administered, as shown in FIG. 3.

### Example 2. Cognitive Function Improvement Effect

To evaluate the cognitive function improvement effect of Yukgunjatang (YGT) in an animal model of cognitive decline, which has been prepared by injecting scopolamine, the Novel Object Recognition Test (NORT) and Passive Avoidance Test (PAT) were carried out. Specifically, Yukgunjatang at a dose of 150 mg/kg was administered orally for 7 days, followed by a co-administration of scopolamine and Yukgunjatang for another 7 days. The normal group (Con) received water and physiological saline, while the control group (SCO) received water and scopolamine, as shown in FIG. 4.

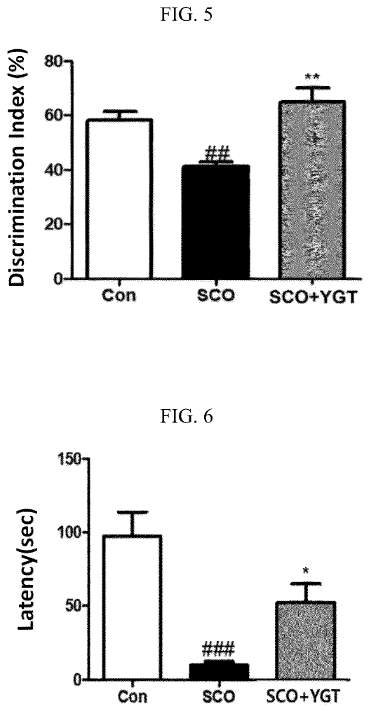

As a result of conducing the Novel Object Recognition Test, as shown in FIG. 5, the discrimination index (i.e., memory index) decreased in the control group (SCO) compared to the normal group (Con), but in the Yukgunjatang treatment group (SCO+YGT), the discrimination index significantly was improved compared to the control group (SCO).

Similarly, in the Passive Avoidance Test, it was found that the latency (i.e., stay time) decreased in the control group (SCO), whereas the Yukgunjatang treatment group (SCO+YGT) showed an increase in latency, as illustrated in FIG. 6.

What is claimed is:

1. A method for improving cognitive function or memory, the method comprising:
administering a composition comprising Yukgunjatang as effective component to a subject in need thereof, the Yukgunjatang comprising Atractylodes rhizoma alba, Gingseng Radix, Hoelen, Glycyrrhizae Radix, Aurantii Nobilis Pericarpium, Pinelliae Rhizoma, Zingiberis Rhizoma, and Zizyphi Fructus.

2. The method according to claim 1, wherein the Yukgunjatang is prepared by boiling a mixture of the Gingseng Radix, the Atractylodes rhizoma alba, the Hoelen, the Glycyrrhizae Radix, the Aurantii Nobilis Pericarpium, the Pinelliae Rhizoma, the Zingiberis Rhizoma, and the Zizyphi Fructus in water.

3. The method according to claim 2, wherein the Yukgunjatang is prepared by boiling a mixture of 0.6 to 2.2 parts by weight of the Gingseng Radix, 0.6 to 2.2 parts by weight of the Atractylodes rhizoma alba, 0.6 to 2.2 parts by weight of the Hoelen, 0.6 to 2.2 parts by weight of the Pinelliae Rhizoma, 0.6 to 2.2 parts by weight of the Aurantii Nobilis Pericarpium, 0.2 to 0.8 part by weight of the Glycyrrhizae Radix, 0.3 to 1.0 part by weight of the Zingiberis Rhizoma, and 0.3 to 1.0 part by weight of the Zizyphi Fructus in water.

4. The method according to claim 1, wherein the composition is in any one formulation selected from powder, granule, pill, tablet, capsule, candy, syrup, and beverage.

5. A method for ameliorating or treating degenerative brain disease, the method comprising:
administering a composition comprising Yukgunjatang as effective component to a subject in need thereof, the Yukgunjatang comprising Atractylodes rhizoma alba, Gingseng Radix, Hoelen, Glycyrrhizae Radix, Aurantii Nobilis Pericarpium, Pinelliae Rhizoma, Zingiberis Rhizoma, and Zizyphi Fructus.

6. The method according to claim 5, wherein the degenerative brain disease is Alzheimer's disease, mild cognitive impairment, and/or dementia.

7. The method according to claim 5, wherein the composition further comprises a pharmaceutically acceptable carrier, an excipient, and/or a diluent.

8. The method of claim 5, wherein the composition is included in an animal feed additive.
